(19) United States
(12) Patent Application Publication
(10) Pub. No.: US 2004/0209934 A1
(43) Pub. Date: Oct. 21, 2004

(54) Protein phosphate inhibitors

(76) Inventors: Adam McCluskey, Mayfield (AU); Jennette Sakoff, Belmont (AU); Stephen Ackland, New Lambton Heights (AU)

Correspondence Address: Ralph A Loren, Lahive & Cockfield, 28 State Street, Boston, MA, 02109, US

(21) Appl. No.: 10/472,805

(22) PCT Filed: Mar. 25, 2002
(86) PCT No.: PCT/AU02/00360

(30) Foreign Application Priority Data
Mar. 23, 2001 (AU) PR 3923

(51) Int. Cl. A61K 31/4178; A61K 31/407; C07D 491/14

(52) U.S. Cl. 514/397; 514/411; 548/311.7; 548/431

(57) ABSTRACT

The present invention relates to modulators of cell cycle regulation and, in particular, to a protein phosphatase inhibitor which can interfere with the cell cycle; processes for the production of the inhibitor; and uses of the inhibitor, in particular in the treatment of disease, such as cancer.

The present invention relates to modulators of cell cycle regulation and, in particular, to a protein phosphatase inhibitor which can interfere with the cell cycle; processes for the production of the inhibitor; and uses of the inhibitor, in particular in the treatment of disease, such as cancer.

### BACKGROUND

Any discussion of the prior art throughout the specification should in no way be considered as an admission that such prior art is widely known or forms part of common general knowledge in the field.

Protein Phosphatases

Phosphorylation of structural and regulatory proteins is a major intracellular control mechanism in cells that is controlled by both protein kinases and protein phosphatases. Such cellular processes including cell cycle progression, glycogen metabolism, gene expression, and phototransduction are modulated by the phosphorylation status of involved proteins. In contrast to the multitude of protein kinases that have been discovered, relatively few protein phosphatases are known. Three major classes of protein phosphatases exist: tyrosine-specific, serine/threonine-specific, and dual-specificity phosphatases. Traditionally, the protein phosphatases that catalyse the dephosphorylation of serine and threonine residues have been classified into four subtypes based on their biological characteristics, sensitivities to specific inhibitors, and substrate specificity (PP1, PP2A, PP2B, and PP2C). The primary amino acid sequences of PP1, PP2A, and PP2B are similar, whereas PP2C is structurally distinct and belongs to a completely different gene family. Other phosphatases including PP4, PP5, PP6 and PP7 have been identified that share amino acid homology with PP1 and PP2A.

Protein Phosphatase Inhibition

Various natural toxins including cantharidin (blister beetles) and fostriecin (Streptomyces pulveraceus-fostreus) have been exploited in the treatment of cancer. Indeed cantharidin is the active component of the reputed aphrodisiac Spanish Fly and has been used as an anticancer agent since 1264ⁱ. We have shown cantharidin to be cytotoxic in ten difference cancer cell lines of ovarian, bone, colon and baematopoietic origin producing IC₅₀ values ranging from 6-18 mM, which is comparable with many conventional cancer drugs (5-flurouracil). Cantharidin is currently in clinical use, however, its nephrotoxicity has prevented its widespread use. Interestingly, cantharidin has been shown to stimulate neutrophil production. This is in contrast with most anticancer drugs whose dose limiting effect is often related to myelosuppression. Fostriecin has also been examined by the National Cancer Institute (ISA) and found to be a potent anticancer agent (IC₅₀ 0.6-50 mM) and subsequently entered Phase 1 clinical trials, however, its structural instability hindered further development.ⁱⁱ More recently, it was discovered that the main target of these drugs is the inhibition of the phosphatase enzymes PP1 and PP2^{iii,iv}.

Cantharidin and fostriecin are members of a structurally diverse group of natural toxins with varying protein phosphatase inhibitory activity (Table 1).^{v} Fostriecin clearly represents the most selective PP2A inhibitor known, while cantharidin (1) is structurally the least complex. Based upon the chemical structure of cantharidin we developed a number of protein phosphatase inhibitors as potent and selective anticancer agents (Table 2). As can be seen in Table 2, the two anhydride modified cantharidin analogues display considerable selectivity towards the colon tumour cell lines, HT29 and WiDr (entries highlighted). However, in most of the reported cases,^{vi} no modification has been possible whilst maintaining potency at either or both PP1 and PP2A.

TABLE 1
Protein phosphatase inhibition (IC₅₀) by naturally occurring toxins.

| | | PP1 | PP2A |
|---|---|---|---|
| 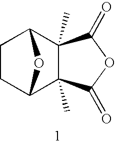 | Okadaic acid Calyculin A Tautomycin Microcystin LR Fostriecin Cantharidin (1) | 42 nM 2 nM 0.3 nM 1.7 nM 131,000 nM 1760 nM | 0.51 nM 0.5 nM 1 nM 0.04 nM 3.4 nM 260 nM |

TABLE 2
Selected anhydride modified cantharidin analogues, PP1 and PP2A inhibition and
cytotoxicity (IC₅₀ = μM, concentration that induces 50% growth inhibition after 72 h exposure).

| | Cytotoxicity in Cancer Cell Lines (IC₅₀ values after 72 h exposure) | | | | | Selectivity of |
|---|---|---|---|---|---|---|
| Cantharidin | HL60 | A2780 | 143B | HT29 | WiDr | PP2A versus PP1 |
| Analogue | Leukaemia | Ovarian | Ostesarcoma | Colon | Colon | inhibition |
| 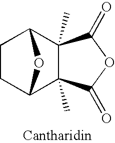 | 10 ± 2 | 10 ± 2 | 10 ± 1 | 6.5 ± 0.5 | 6.1 ± 0.5 | 5.5 |
| 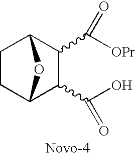 | 270 ± 15 | 110 ± 10 | 450 ± 50 | 15 ± 5 | 33 ± 6 | 10.3 |
| 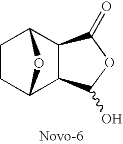 | 393 ± 103 | 333 ± 55 | 454 ± 50 | 14 ± 0.3 | 15 ± 3 | 5.0 |

Phosphorylation and Cell Cycle Progression

As with most anticancer agents the cytotoxicity of cantharidin and fostriecin is mediated via modifications of the cell cycle. The cell cycle is regulated by an intricate phosphorylation network involving an interplay between kinase and phosphatase activity.^{vii} Both PP1 and PP2A have been shown to control key signal transduction mechanisms involved in cell cycle progression, with PP1 and PP2A often classified as negative regulators of the cell cycle.^{viii,ix,x,xi} This negative regulation involves the inactivation of the retinoblastoma protein (pRb), and cyclin dependent kinases (cdk). The latter can be achieved by directly dephosphorylating cdks or indirectly by influencing upstream and downstream kinases/phosphatases. Other mitotic events are also controlled by PP1 and PP2A including chromosome condensation, nuclear membrane disintegration, reorganisation of cytoplasmic microtubules, spindle formation, chromatid separation, nuclear membrane reassembly and cytokinesis.^{xii}

The negative role of protein phosphatases in cell cycle control is further exemplified by the fact that; (a) several natural PP1 and PP2A inhibitors (okadaic acid) are tumour promoters (b) several viral proteins such as the polyoma middle T and SV40 small T antigen are potent PP2A inhibitors which stimulate the growth of senescent cells, (c) the overexpression of the endogenous protein phosphatase inhibitor (SET) is thought to be involve in the formation of Wilms Tumour and (d) protein phosphatase inhibitors stimulate movement through the cell cycle resulting in premature mitosis (fostriecin, okadaic acid, calyculin A).^{xiii,xiv,xv}

There is a need for alternative cancer treatments. Further, there is a also a requirement for alternative, and preferably potent, PP1 and PP2 inhibitors which modulate the cell cycle and thus can be used as pharmaceuticals to combat disease, in particular cancer.

It is an object of the present invention to overcome or ameliorate at least one of the disadvantages of the prior art, or to provide a useful alternative.

### BRIEF DESCRIPTION OF THE FIGURES

FIG. 1: Structure of cantharadin (1) and tautomycin (2).

FIG. 2: (a) Crystal structure of microcystin-LR bound to PP1; (b) Computer model (Cerius2-LigandFit) cantharidic acid bound to PP1.

FIG. 3: Panels 3a and 3c illustrate the overlap of enantiomers maximising interactions with the imide ring and histidine a-carbons. Panels 3b and 3d illustrate the preferred docked conformations of 14/15 with PP1. There are noticable differences in the orientations of the histidine ring and the carboxylate.

FIG. 4: Overlay of 14, 15 cantharidic acid, Microcystin-LR and PP1.

FIG. 5: (a) Computer model of NOVO-20 bound in the active site of PP1; (b) Computer model of NOVO-21 bound in the active site of PP1

FIG. 6: Cell Cycle analysis (% distribution) of HCT116 cells treated with Cantharidin (25 μM) for 6 and 24 h. The same response was observed in HT29 and L1210 cells. Note the significant increased S-phase population after 6 h (4.6 fold increase in ³H thymidine uptake) and the subsequent induction of G₂/M arrest. **P<0.05, **P<0.01.

FIG. 7: Results of cytoxicity studies performed on different cell types using the compounds of the invention.

### SUMMARY OF THE INVENTION

While protein phosphatase inhibition stimulates the cell cycle, excessive or premature cell cycling is lethal via the same mechanism. The forcing of cells through the cell cycle prematurely (although perhaps counter-intuitive compared with the current approaches) can, therefore, be used in the treatment of cancer.

A new series of cantharidin analogues has been identified. It has unexpectedly been found that some of these new analogues are potent inhibitors of PP1 and PP2.

Accordingly, in a first aspect, the present invention provides a protein phosphatase inhibitor wherein the inhibitor is a cantharimide or an analogue thereof.

Preferably, the phosphatase is a serine/threonine specific phosphatase and most preferably, it is PPI or PP2A. Preferably, the protein phosphatase inhibitor is cell permeable.

According to a second aspect, the present invention provides a compound of formula I

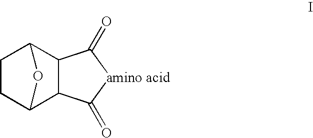

or an analogue, derivative or variant thereof.

The amino acid may be a D- or L-amino acid or an analogue thereof and is preferably selected from the group consisting of D-Alanine, L-Alanine, D-Phenylalanine, L-Phenylalanine, D-Leucine, L-Leucine, D-Isoleucine, L-Isoleucine, D-Tryptophan, L-Tryptophan, D-Histidine, L-Histidine, D-Tyrosine, L-Tyrosine, D-Glutamine and L-Methionine. More preferably, the amino acid is D-Tryptophan, L-Tryptophan, D-Histidine, L-Histidine, D-Tyrosine, L-Tyrosine, D-Glutamine or L-Methionine and most preferably, the amino acid is D-Histidine or L-Histidine.

Preferably, the compound is a cell cycle modulator and more preferably, the compound stimulates the cell cycle. In a preferred embodiment, the compound is a protein phosphatase inhibitor and most preferably, the compound is an inhibitor of PP1 and/or PP2A.

According to a third aspect, the invention provides a method of preparing a compound according to the first or second aspect, wherein the method includes combining a compound of formula II

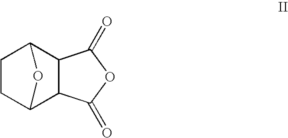

or an analogue, derivative or variant thereof, with an amino acid or an analogue, derivative or variant of an amino acid.

The specific amino acid to be used will, of course, be determined by the required compound.

Preferably, the method includes combining the compound of formula II with the amino acid in the presence of an amine. Preferably the amine is a tertiary amine and most preferably it is Et₃N.

More preferably, the method further includes combining the compound of formula II with the amino acid in the presence of an organic solvent and preferably the organic solvent is PhCH₃.

The method may be carried out at any suitable temperature as determined by the skilled addressee. Preferably, the temperature is between 15 and 300° C. For example, the reaction may be carried out at room temperature, or, in order to reduce the time required, it may advantageously be carried out between 100° C. and 250° C. In certain embodiments the reaction may be carried out at approximately 200° C. Generally, as the skilled addressee will recognise, the lower the temperature, the longer the reaction time.

Preferably, the compound prepared according to the method of the third aspect is further purified. In a preferred embodiment the compound may be washed, preferably with NaHCO₃, extracted, preferably with CH₂Cl₂ and acidified, preferably with concentrated HCl. The resultant compound may advantageously be extracted with an organic solvent, such as ethyl acetate and may be further purified by chromatography, such as column chromatography.

According to a fourth aspect, the present invention provides a compound of formula III

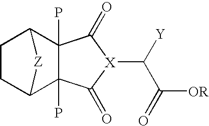

wherein Z is O, NH or NR;

X is CH or N;

wherein P may be absent or may be methyl;

Y is (CH₂)ₙ W;

wherein W is any ionisable residue and n can be any integer from 1 to 8.

Preferably, the compound is

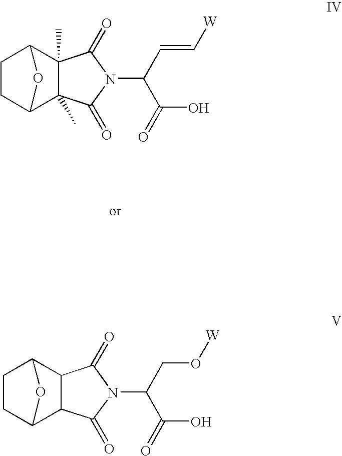

According to a fifth aspect, the present invention provides a compound of formula III as defined above when used to modulate the cell cycle of a cell.

In a preferred embodiment, Z is O, X is N and Y is —CH₂OW wherein W is an amino acid or an analogue or derivative of an amino acid. Preferably the amino acid is D-Histidine or L-Histidine.

In another preferred embodiment, Z is O, X is N and Y is —CH₂═CHW wherein W is an amino acid or an analogue or derivative of an amino acid. Preferably the amino acid is D-Histidine or L-Histidine.

According to a sixth aspect, the present invention provides a pharmaceutical composition including a compound according to the invention. The pharmaceutical may be administered by any suitable route, preferably intravenously. Pharmaceutically acceptable adjuvants, carriers and/or excipients may be used. Suitable such pharmaceutically acceptable substances are those within the knowledge of the skilled person.

According to a seventh aspect, the present invention provides use of a compound according to the invention in the manufacture of a medicament. Preferably, the medicament is useful for the treatment of cancer. Preferably the cancer is leukemia, ovarian, colon or kidney cancer.

According to an eighth aspect, the present invention provides a method of treating a disorder or a disease including administering a compound or a pharmaceutical composition according to the invention. Preferably, the disease is cancer. Preferably, the cancer is leukemia, ovarian, colon or kidney cancer.

In certain embodiments, the method of treatment may be combined with a further treatment. Preferably, the method of treatment renders treated cells more sensitive to the further treatment. Typically, the further treatment will be a treatment for cancer. Most preferably, the further treatment may be selected from the group consisting of: radiation, cisplatin, 5-flurouracil, methotrexate, thymitaq and taxol treatment.

According to a ninth aspect, the present invention provides a method of regulating cell cycle progression including exposing a cell to a compound according to the invention. Preferably, one or more of the cell cycle checkpoints are abrogated by the compound.

Preferably, the cell is a mammalian cell although it will be clear that the invention is not confined to a specific cell type and could be used for any suitable cell type of any origin including bacteria, yeast, lower and higher eukaryotes.

In a preferred embodiment, the cell is a human cell and most preferably, the cell is a neoplastic or pre-neoplastic cell and most preferably, is a cancer cell. Preferably, the cell is selected from the group of: a murine leukaernia cell, a human leukaemia cell, a human ovarian cancer cell, a cisplatin resistant cell, a human colon carcinoma cell, a human oesteosarcoma cell and a human kidney tumour cell. Most preferably, the cells is a cell selected from those cell types known as L1210 (murine Leukaemia, p53 mt), HL60 (human leukaemia, p53 nul), A2780 (human ovarian carcinoma, p53 wt), ADD (cisplatin resistant A2780 cells, p53 mt), HCT116 (human colon carcinoma, p53 wt), SW 480 (human colon carcinoma, p53 wt), WiDr (human colon carcinoma, p53mt), HT29 (human colon carcinoma, p53 mt), 143BTK-(human oesteosarcoma) and G-401 (human kidney, Wilms tumour) cells.

According to a tenth aspect, the present invention provides a method of sensitising a patient to a cancer treatment, comprising administering to the patient an effective amount of a compound of the invention.

Actual dosages of the compounds and pharmaceutical compositions of the invention to be delivered to patients may be varied so as to obtain an amount of the active ingredient which is effective to achieve the desired response for a particular patient, composition and mode of adrinistration. The dosage level can be readily determined by the physician in accordance with conventional practices and will depend upon a variety of factors including the activity of the particular compound of the invention to the administered, the route of administration, the time of administration, the rate of excretion of the particular compound employed, the age, sex, weight, condition, general health and prior medical history of the patient being treated, and like factors well known in the medical arts.

According to an eleventh aspect, the present invention provides a method of preparing a compound of formula III as defined above including combining

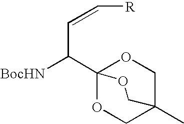

wherein R is an ionisable residue

with cantharidin or an analogue or derivative thereof.

According to a twelfth aspect, the present invention provides a method of preparing a compound of formula III as defined above including combining

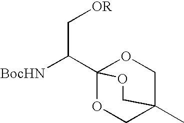

wherein R is an ionisable residue

with cantharadin or an analogue or derivative thereof.

It will be clear to skilled addresses that amino acid analogues may be used in the synthesis of the compounds of the invention. Many amino acid analogues are commercially available and the skilled addressee will also be aware of methods for the generation of such analogues. For example, histidine analogues with modified sidechains such as

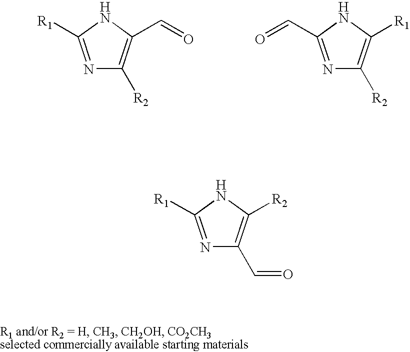

are useful in the present invention.

Further, it will be clear to the skilled addressee that by choice of synthetic methodology, e.g. Horner-Emmons-Willing or Julia-olefination conditions, the conformation of the compounds of the present invention may be manipulated.

### DESCRIPTION OF THE INVENTION

A preferred embodiment of the invention will now be described by way of example only.

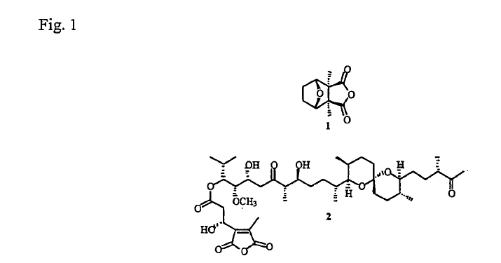

Cantharidin (1) and tautomycin (2) (FIG. 1) are both known to bind in the active sites of both PP1 and PP2A in the ring-opened dicarboxylic acid form. However, we have recently shown that a series of ring-opened cantharidin analogues with only one free carboxylate, see Table 1, not only retained inhibitory activity against PP1 and PP2A but increased slightly the selectivity towards PP2A. As a result of this work, we have been forced to re-evaluate our understanding of the inhibition of PP1 and PP2A by cantharidin analogues. The first step in our re-evaluation was a closer examination of the crystal structure of PP1 and of a molecularly modeled structure of PP2A.

TABLE 1
Inhibition of PP1 and PP2A by ring-opened cantharidin analogues.

| 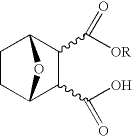 | PP1 IC₅₀μM^{a} | PP2A IC₅₀μM^{a} | PP2A/ PP1 |
|---|---|---|---|
| R = CH₃ | 4.71 | 0.41 | 11.5 |
| R = CH₃CH₂ | 2.96 | 0.45 | 6.5 |
| R = CH₃CH₂CH₂ | 4.82 | 0.47 | 10.2 |

^{a}Average of three experiments in triplicate

Our modeling analysis (data not shown) suggested that these ring-opened analogues bind in the active site and that the sole carboxylic acid residue still binds in the active site of PP1 and PP2A. The alkyl chain of the ester is proximal to an acidic groove in the general vicinity of the protein's active site. The major amino acids in this groove are acidic in nature, and consequently we believed that compounds possessing the anhydride bridge of cantharidin (or the demethylated norcantharidin), a single carboxylic acid group and a basic residue would give rise to a new class of compounds with the ability to inhibit PP1 and PP2A. The following describes a new class of cantharidin analogues that inhibit both PP1 and PP2A.

Without being bound by theory, it appears that these new protein phosphatase inhibitors mediate their effects via the cell cycle and the abrogation of cell cycle checkpoints. Such phosphatase inhibitors would not only be lethal as single agents but also potentiate the cytotoxicity of more conventional agents.

### Chemical Synthesis of Novel Cantharidin Analogues

Design and Synthesis of a New Class of Protein Phosphatase Inhibitors Based Upon the Chemical Structure of the Known PP1 and PP2A Inhibitor Cantharidin

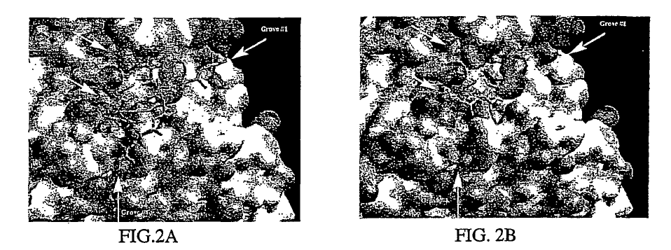

FIG. 2a shows the partial crystal structure of PP1 with microcystin-LR, a cyclic heptapeptide, present within the active site (co-crystallised). The active site of PP1 is located on the surface of the protein, as it is with PP2A (no crystal structure has been reported, although a homology-modelled structure has been developed).^{xvi} Microcystin-LR comprises a cyclic core and a hydrophobic tail (the novel amino acid Adda). Important features include: i) the binding of Adda along Groove #1 (also known as the hydrophobic groove). Modification of this residue is detrimental to binding; ii) the Arg-sidechain of micro cysin-LR is proximal to, but not present in, Groove #2. Although Quinn^{xvii} has shown that the Arg is not necessary for binding, our analysis suggests that a modified Arg-residue, better able to occupy Groove #2 should influence binding at both PP1 and PP2A (data not shown); iii) the cyclic peptide core is proximal to two other surface groves annotated: Groove #3 and Groove #4. In the latter instance the Leu-sidechain of microcystin-LR is proximal to, but not present in, Groove #4.

FIG. 2b shows the same view for cantharidic acid (the bound form of cantharidin). Significantly cantharidic acid does not present any residues into Grooves #1-4.

It is known that the lack of a hydrophobic tail able to access Groove #1 is responsible for the 1000-fold decrease in activity against PP1 and PP2A observed with cantharidin (μM versus nM^{xviii}). When modelled in this fashion our previously reported anhydride modified cantharidin analogues present the ester (NOVO-4) and ether (NOVO-9) groups (the “R”-groups are shown diagrammatically in scheme 1) towards, but not fully into Groove #1.^{xix}

Targeting Grooves #2-4. The Development of Potent, Selective PP1/PP2A Inhibitors, and Potent Anticancer Agents.

Models describing the interaction of the known toxins with both PP1 and PP2A have been developed (FIG. 2a). These toxins, e.g. the Microcystins, nodularins, calyculins, tautomycin and okadaic acid all have a hydrophobic tails that extend along Groove #1.

None of the known toxins access Grooves #2, 3 or 4. To date the potential significance of the other Grooves has been neglected, possibly due to the synthetic complexity of the major toxins. Typically >50 synthetic steps and 1-2 years laboratory effort are required to affect the necessary modifications.

Cantharidin and its demethylated analogue, norcantharidin, are more promising compounds which, until now have largely thwarted attempts, by structural modification, to improve either potency or selectivity. However, we have discovered a new class of cantharidin analogues, the cantharimides, that can be significantly modified and still retain potency against PP1 and PP2A. This is the first time cantharidin has been modified and retained activity against PP1 and PP2A.

Further, we are able to synthesise the cantharimides in a single synthetic step that furnishes us with gram quantities of pure cantharimides in one day (Scheme 1). This represents a significant breakthrough in the development of protein phosphatase inhibitors.

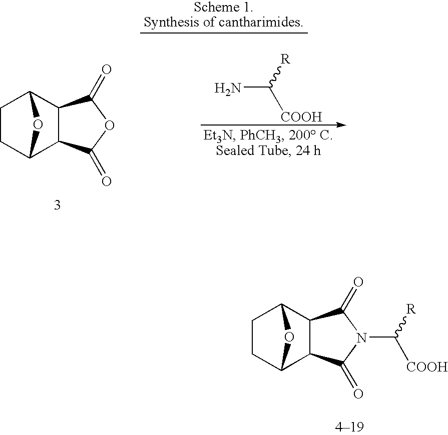

### EXAMPLE 1

Synthesis of Cantharimides

Typically, the cantharimide analogues were synthesised in moderate to good yield by a modified Gabriel synthesis commencing from readily available norcantharidin (3). In a typical synthesis 168 mg of 3 was placed in a thick walled glass pressure vessel, followed by anhydrous toluene (10 mL), 1.0 equivalents anhydrous Et₃N and 1.0 equivalents of unprotected amino acid (either D or L).

In a typical synthesis, D-Phe (165 mg, 1 mmol) was placed in a thick walled glass pressure vessel along with anhydrous toluene (5 mL), anhydrous triethylamine (101 mg, 140 mL, 1 mmol) and norcantharidin (168 mg, 1 mmol) and a magnetic stirrer. The heterogeneous mixture was degassed, the tube sealed and the mixture heated to 200° C. behind a safety shield. After ca 16 hr, the homogeneous mixture was cooled to room temperature and opened cautiously. The contents were taken up in either ethylacetate or dichloromethane (25 mL) and washed with saturated NaHCO₃. The aqueous layer was then extracted with dichloromethane (2×10 mL) and acidified to pH2 by the dropwise addition of 6 M HCl, re-extracted with dichloromethane (2×10 mL), dried over Na₂SO₄. The solvent was removed in vacuuo to yield an oil. Purified by chromatotrom plate (solvent system: EtOAc: Hexanes 19:1). ¹HNMR (300 MHz, CDCl₃): d 1.94 (CH₂), 2.92 (CH), 3.33 (CH₂), 4.10 (CH), 4.84 (CH), 6.9-7.30 (Ph), 9.81 (CO₂H); ¹³C NMR (75 MHz, CDC13): d 25.3, 29.15, 45.02, 51.82, 76.58, 126.17, 128.56, 129.38, 137.89, 170.08, 175.81.

Work-up comprised a NaHCO₃ wash, extraction with CH₂Cl₂, followed by acidification with concentrated HCl, and finally extraction with ethyl acetate. The crude products were then purified by column chromatography. Typical yields are shown in Table 2 below.

The simplicity of the chemical synthesis, and the ready availability of both D- and L-amino acids allowed examination of the effect of the different stercoisomers.

### EXAMPLE 2

Screening for Ability of the Compounds to Inhibit PP1 and PP2

The cantharimides 4-19 were screened for their ability to inhibit PP1 and PP2A.

Protein phosphatase assays were carried out essentially as described (Collins, E.; Sim, A. T. R.; Methods in Molecular Biology 1998, 93, 79-102) using [³²P]-glycogen phosphorylase a as substrate and recombinant PP1 (Bernt, N. Methods in Molecular Biology, 1998, 93, 67-78) or partially purified (chicken skeletal muscle) PP2A catalytic subunits (Mackintosh, C. In Protein Phosphorylation: A Practical Approach, Ed D G Hardie, IRL, 1993). Briefly, enzyme activity was measured at 30 ° C. in a buffer (final volume of 30 mL) containing 50 mM Tris-HCl (pH 7.5), 1 mM EGTA, 0.1 mM EDTA, 5 mM Caffeine, 0.1% mercaptoethanol, 0.3 mg/mL BSA. The concentration of PP1 or PP2A used was such that the reaction was limited to 15% dephosphorylation to ensure linearity. The reaction was started with the addition of 30 mg [³²P]-glycogen phosphorylase a and terminated after 20 minutes by the addition of 100 mL ice-cold 70% TCA. After 10 minutes on ice the sample was centrifuged and a 100 mL aliquot of the supematant was removed for scintillation counting of the [³²p] released during the reaction. Data is expressed as the IC₅₀ concentration of the compound, which represents the concentration of compound required to produce 50% inhibition of protein phosphatase activity relative to a control (absence of inhibitor) incubation (100% activity).

Cantharidin (1), and norcantharidin (3) were included as internal standard to ensure the relative validity of our protocol, and to allow the effects of differing assay conditions to be standardised.

The results of the phosphatase inhibition study are shown in Table 2. We note that analogues 4-11 showed effectively no inhibitory effects at either PP1 or PP2A. Although 4 did show marginal activity at PP2A. However, the introduction of a bulky aromatic group, such as in 12 and 13 resulted in a significant increase, although still poor, in protein phosphatase inhibition, (12: PP1 IC₅₀═770±146 mM; PP2A IC₅₀═157±33 mM and 13: PP1 IC₅₀═312±153 mM; PP2A IC₅₀═105±22 mM). In terms of the active analogues there appeared to be no overall discernable trend in the effect of a particular stereoisomer on phosphatase inhibition. In this context the L-isomer of Tryptophan was more active at inhibiting both enzymes than the D-isomer, while the reverse trend was observed for the Tyrosine analogue, and no significant stereoisomer effects were observed for the Histamine analogue. It is interesting to note that these stereoisomer effects were similar for both enzymes, with the exception of Alanine, where the D-isomer showed greater inhibition over the L-isomer but only for PP2A.

TABLE 2
Inhibition of protein phosphatases 1 and 2A by compounds 1, 3 and
cantharimides 4-19

| | Parent Amino | Yield | PP1 Inhibition | PP2A Inhibition | PP2A |
|---|---|---|---|---|---|
| Compound | Acid | (%) | IC₅₀ (μM)^{a} | IC₅₀ (μM)^{a} | Selectivity |
| 1 | 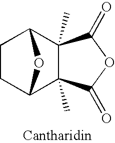 | — | 3.6 ± 0.42 | 0.36 ± 0.08 | 10 |
| 3 | 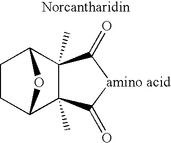 | — | 5.31 ± 0.36 | 2.9 ± 1.04 | 1.83 |
| 4 | D-Ala | 48 | >1000 | 150 ± 85 | ˜6 |
| 5 | L-Ala | 53 | >1000 | >1000 | — |
| 6 | D-Phe | 50 | >1000 | >1000 | — |
| 7 | L-Phe | 57 | >1000 | >1000 | — |
| 8 | D-Leu | 76 | >1000 | >1000 | — |
| 9 | L-Leu | 70 | >1000 | >1000 | — |
| 10 | D-Ile | | >1000 | >1000 | — |
| 11 | L-Ile | 64 | >1000 | >1000 | — |
| 12 | D-Tryptophan | 31 | 770 ± 146 | 157 ± 33 | 4.9 |
| 13 | L-Tryptophan | 57 | 312 ± 153 | 105 ± 22 | 3.0 |
| 14 | D-His | 8 | 3.22 ± 0.7 | 0.81 ± 0.1 | 4.0 |
| 15 | L-His | 14 | 2.82 ± 0.6 | 1.35 ± 0.3 | 2.1 |
| 16 | D-Tyrosine | 21 | 101 ± 34 | 112 ± 10 | 0.9 |
| 17 | L-Tyrosine | 74 | 570 ± 330 | 245 ± 65 | 2.3 |
| 18 | D-Glutamine | 16 | 95 ± 5 | 32 ± 8 | 3.0 |
| 19 | L-Methionine | 32 | >1000 | >1000 | — |

^{d}Average of three experiments in triplicate

Other trends also emerged from within the more potent cantharimides. In addition to confirming that a basic amino acid residue would facilitate inhibition of PP1 and PP2A, it was noted that those cantharidimides possessing an easily ionisable group also showed activity.

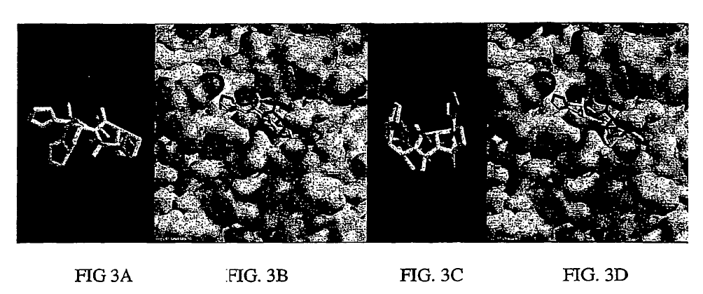

Of all the cantharimides reported in the table, only 14 and 15 are essentially equipotent with cantharidin. Surprisingly there is almost no differentiation in binding. This is an unexpected result, given the relative spatial orientation of the His-sidechain, carboxylic acid and tricyclic core in each of the enantiomers (see FIG. 3).

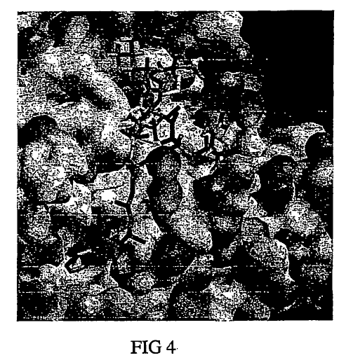

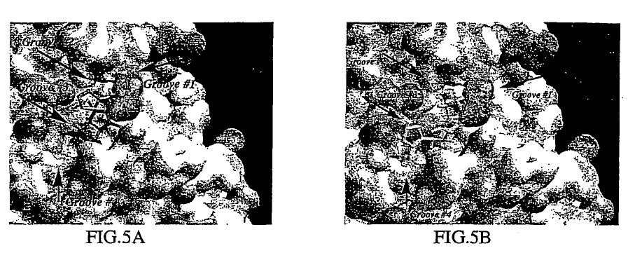

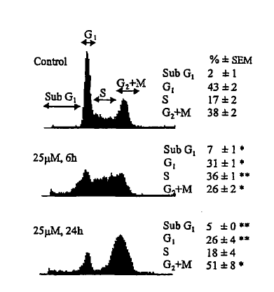

Our modelling analysis (Cerius2—LigandFit) results indicated two, high scoring, possible binding modes that explain our observed lack of selectivity. Only data for 14/15 and PP1 is shown, in all instances 14/15 also bind in the same location as cantharidic acid (see FIG. 4). Panels 3a and 3c illustrate the overlap of enantiomers maximising interactions with the imide ring and histidine a-carbons. However this results in poor carboxylate alignment and places the 7-Os on opposite sides. This should result in a difference in inhibition of PP1 and PP2A, as it is known that the 7-O is crucial for inhibition (at least of PP2A). Panels 3b and 3d illustrate the preferred docked conformations of 14/15 with PP1, however there are noticable differences in the orientations of the histidine ring and the carboxylate.

Surprisingly, in this instance 14 and 15 are presented to the enzyme in completely different orientations, essentially head-first or feet-first. Although this possibility at first appears contrary to expectations, it does allow for presentation of similar features in similar positions to the enzyme. Notable the carboxylate of 14 and the 7-O of 15 present themseleves in the same area of the protein and vice-versa.

Conformational analysis and overlays were performed using CERIUS2.4.2 (MSI, San Diego, 2000 release) and the AM1 electrostatic potential surfaces were generated using the minimum energy conformers (from CERIUS2.4.2) in MacSpartanPro.

All cantharirnides displaying inhibitory action at either PP1 or PP2A present essentially an identical three-dimensional structure to the enzyme. However, upon consideration of the electrostatics of each analogue, we note that there are significant variations with the most active His analogues presenting a more negative surface to the active site. This is consistent with our initial belief that the introduction of a basic amino acid side chain would facilitate interaction with the known acidic groove proximal to the active site.

Hence, in summnary, compounds which are a hybrid of norcantharidin and the amino acids D/L-histidine (named NOVO-20 & NOVO-21 respectively) are potent inhibitors, exhibiting protein phosphatase inhibition equipotent with cantharidin, and a similar cytotoxicity profile (Table 3).

TABLE 3
Summary of protein phosphatase inhibition and cytotoxicity data
obtained for NOVO-30 and NOVO-21.

| PP2A | | | | | |
|---|---|---|---|---|---|
| Inhibition | PP1 Inhibition | Cytotoxicity in Selected Cell-Lines | | | |
| (μM) | (μM0 | (IC₅₀ μM) | | | |
| NOVO-20 | | L1210 | 6 ± 1 | HT29 | 28 ± 9 |
| 0.81 ± 0.1 | 3.22 ± 0.7 | HL60 | 21 ± 13 | SW480 | 32 ± 12 |
| NOVO-21 | | L1210 | 30 ± 14 | HT29 | 28 ± 10 |
| 1.35 ± 0.3 | 2.82 ± 0.6 | HL60 | 55 ± 24 | SW480 | 61 ± 30 |

We were initially surprised by our pilot data of no protein phosphatase selectivity with the pure enantiomers, as we have anticipated some form of differential binding to the protein via interaction with Groove #2. The computer modelled interactions of NOVO-20 and NOVO-21 with PP1 (FIG. 5) and show great contrast in binding modes of these enantiomers.

FIG. 5a shows the computer model of NOVO-20 and PP1 with the terminal imidazole ring at the apex of Grooves 2 & 3. Similarly, FIG. 5b shows the computer model of NOVO-21 with the terminal imidazole ring at the apex of Grooves #3 & 4. As can be seen in both figures, neither of the terminal imidazole rings fully engages Grooves #2, 3 or 4.

It will be clear to the skilled addressee, in light of the above that selective modification of the terminal imidazole with NOVO-20, and lengthening the “spacer-arm’ will allow access to Groove #2 and/or Groove #3 resulting in an increase in PP1/PP2A selectivity. Similar modifications of NOVO-21 will facilitate access to Grooves #3 and/or 4 (see schemes 3 & 4). Protein phosphatase selectivity (PP1 and/or PP2A) will arise as a consequence of the known differential contact points in Grooves #2-4 in these proteins.

Our modelling analysis has shown that the proposed analogues, schemes 1 & 2, will allow access to Grooves #2-4. These grooves offer differential points of contact in PP1 and PP2A. Our synthetic approaches have been designed so that we can accomplish selective access to each of these Grooves in turn, and in all possible combinations. In particular our modelling predicts:

That the introduction of a basic residue will access Groove #2 (lined with acidic amino acids).

That Grooves #2-4 display differential points of contact in PP1 and PP2A allowing for the development of new analogues that selectively interact with either PP1 or PP2A as a result of different amino acid sequences in the relevant regions.

That accessing Grooves #2-4 will allow unprecedented development of low molecular weight ligands with high potency (nM) and selectivity (>100-fold) for either PP1 and/or PP2A.

### EXAMPLE 3

Chemistry for Targeting Grooves#2-4:

It has been shown that the cantharimides are potent PP1 and PP2A inhibitors. Modelling analysis has enabled the design of selected cantharimide analogues with the potential to maximise favourable interactions with the active site of PP1 (and PP2A). Accordingly, we have designed synthetic protocols that take advantage of: readily available optically pure starting materials, e.g. D- and L-Serine, as well as established synthetic methodologies to facilitate rapid development of the targets required for validation of our hypothesis, and assist in the development of new therapeutic agents. The skilled addressee will be aware of simple methodologies which facilitate the extension of the spacer arm (see scheme 2) for improving the access of the terminal imidazole ring (of NOVO-20 & 21) into Grooves #2 and/or 3 and/or 4 (dependent on imidazole ring substitution pattern. Synthesis of any desired analogues is possible as shown in Scheme 2 below.

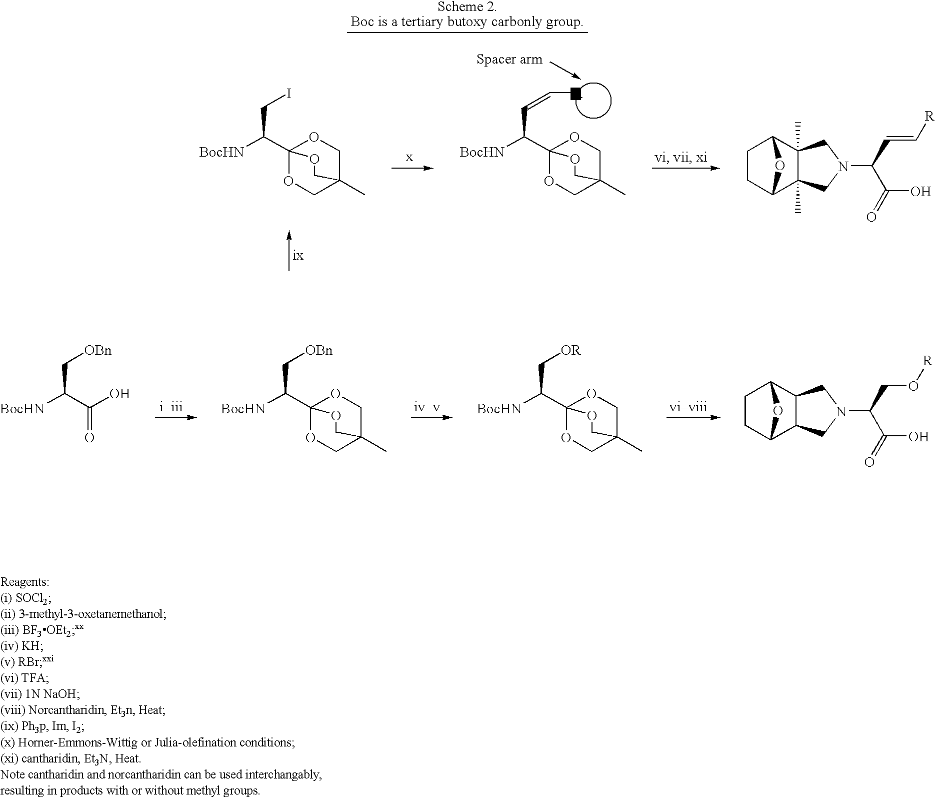

Such routes (Scheme 2) are flexible because they allows the synthesis of analogues possessing the O-atom, as found in scrine and simple alkylation, or the removal of this O-atom and the implementation well established synthetic transformations.

### EXAMPLE 4

Synthesis of Sidechain Groups (R)

It will be clear to the skilled addressee that histidine analogues with modifed sidechains are easily obtained via commercially available substituted imidazoles, such as those illustrated in scheme 3. This makes them ideal starting materials.

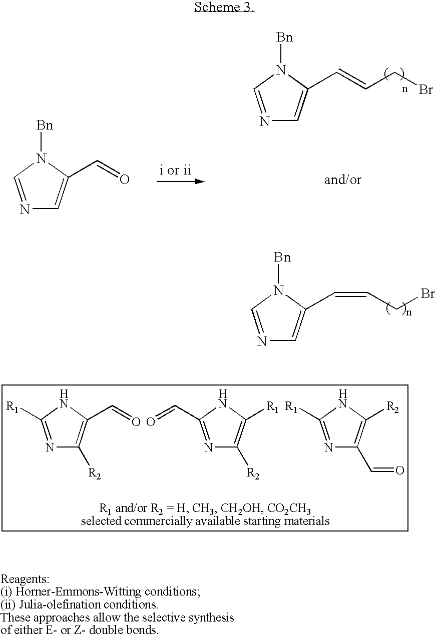

Additionally, choice of synthetic methodologies, allows stereochemical control of the resultant double bond. Such control in turn allows subtle manipulation of the orientation of the final target coumpounds (shown schematically in Scheme 4) and thus a deal of fine control for their interaction with both PP1 and PP2A.

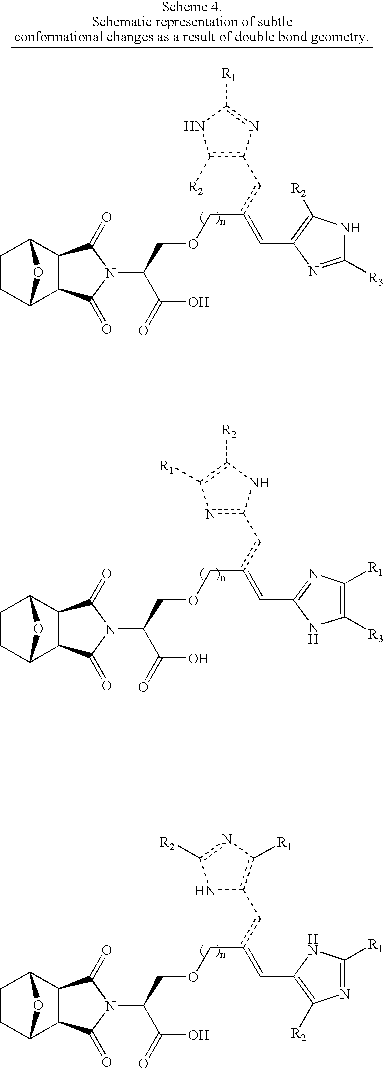

It will be clear to the skilled addressee that the synthetic chemistry portion of generate discrete libraries of cantharidin analogues (eg. based on NOVO-4, 9, 20 and 21) can be rapidly screened for PP1 and PP2A activity.

### EXAMPLE 5

Testing the Biologicalproperties of the Compounds in Cell Line Models of a Number of Human Cancers.

Cytotoxicity

Compounds showing protein phosphatase inhibition were screened for their ability to inhibit the growth of various cell lines. The cell lines chosen for study include both normal (non-cancer) and tumour-derived cell lines that represent both haematopoietic and solid tumor types with varying cell cycle control mechanisms (ie p53 status); CCD-18Co (normal human colon), L1210 (murine leukaemia, p53 mt), HL60 (human leukaemia, p53 nul), A2780 (human ovarian carcinoma, p53 wt), ADDP (cisplatin resistant A2780 cells, p53 mt), HCT116 (human colon carcinoma, p53 wt), SW480 (human colon carcinoma, p53 wt), WiDr (human colon carcinoma, p53 mt), HT29 (human colon carcinoma, p53 mt) and 143BTK-(human oesteosarcoma) cells. In addition to these cell lines,it will be clear to the skilled addressee that the inhibitors could also be used to screen G401 cells (human kidney, Wilms tumour), as evidence suggests that this tumour type may have an inherently reduced protein phosphatase activity.

The MTT assay can be used assess cytotoxicity. This assay determines cell viability by the ability of mitochondrial dehydrogenase to produce formazan crystals from 3-(4,5-dimethylthiazol-2-yl)2,5-diphenyltetrazolium bromide. The viable cell number/well is directly proportional to the production of formazan, which following solubilization, can be measured spectrophotometrically (540 nm). This technique produces a “dose-response” curve from which an IC₅₀ value (concentration that inhibits growth by 50%) is calculated. This technique is also used by the NCI(National Cancer Institute, USA) to screen for new anticancer agents. The MTT assay may be performed 72 h after exposure of the cells to varying doses of the phosphatase inhibitors. Since these agents are likely to interfere with the cell cycle it is important to test the cytotoxicity for at least two-three cell divisions. Cytotoxicity can, of course, be correlated with protein phosphatase inhibition.

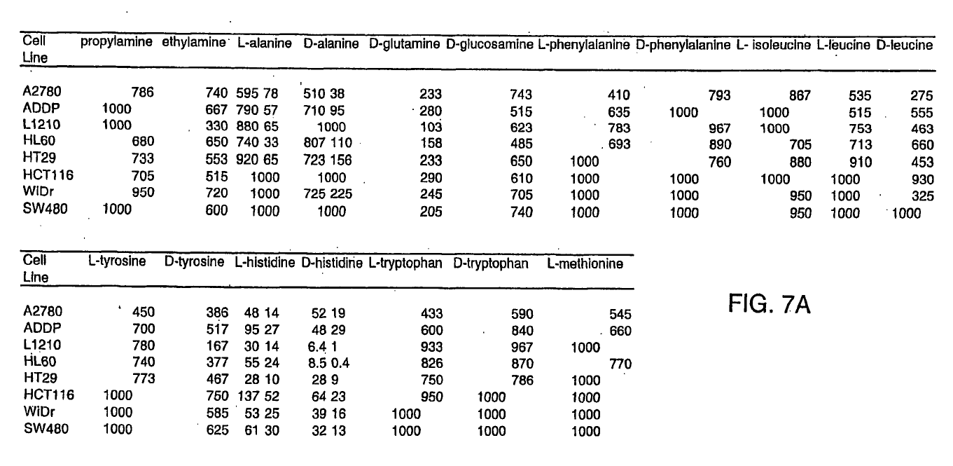

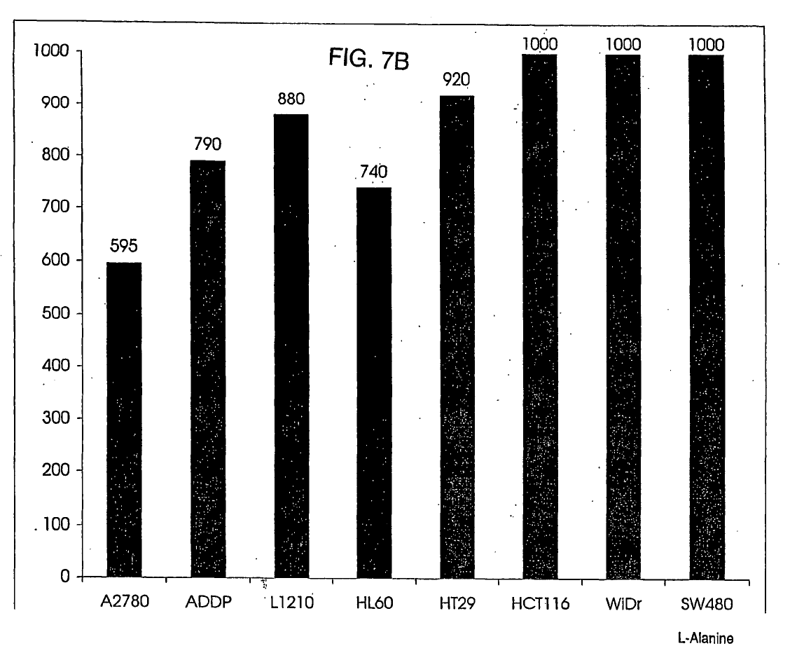

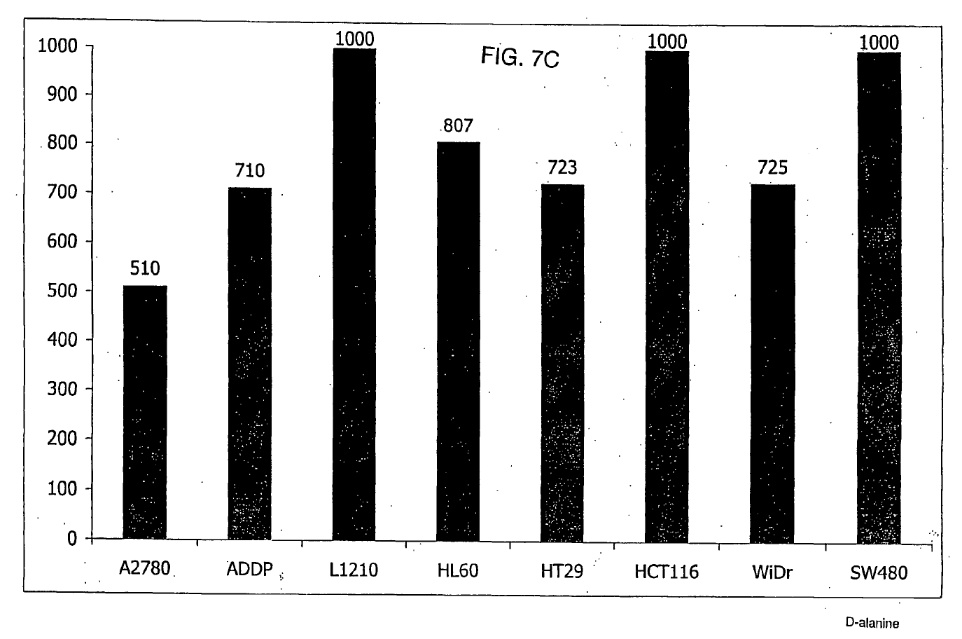

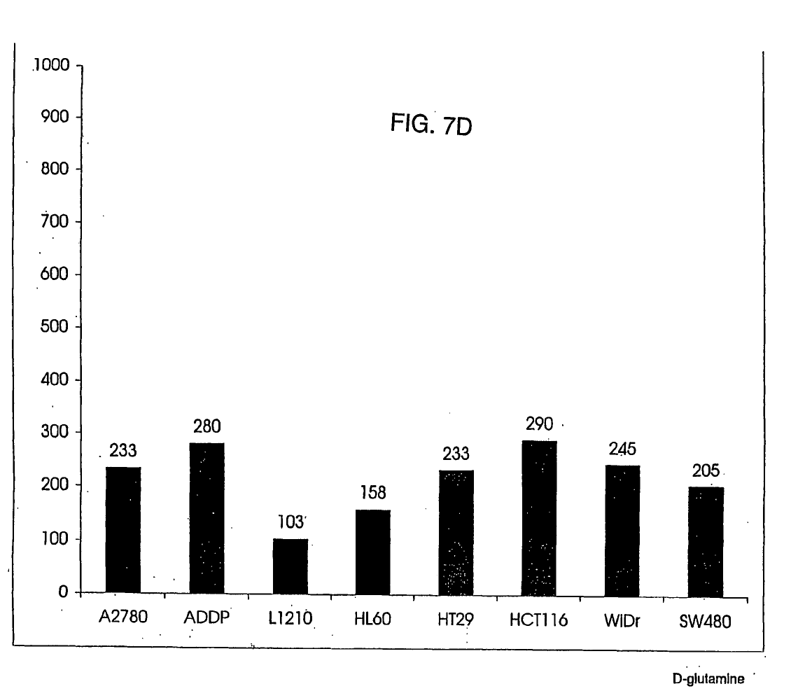

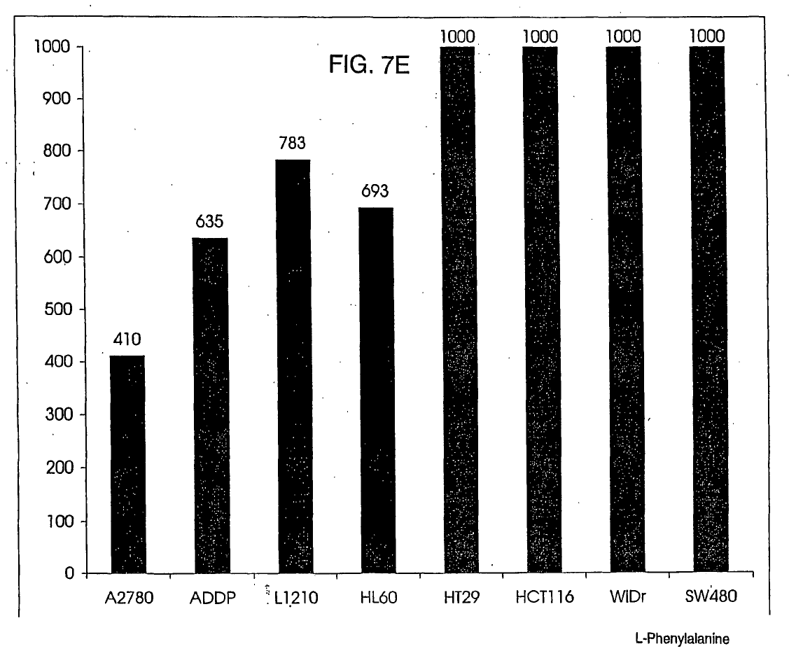

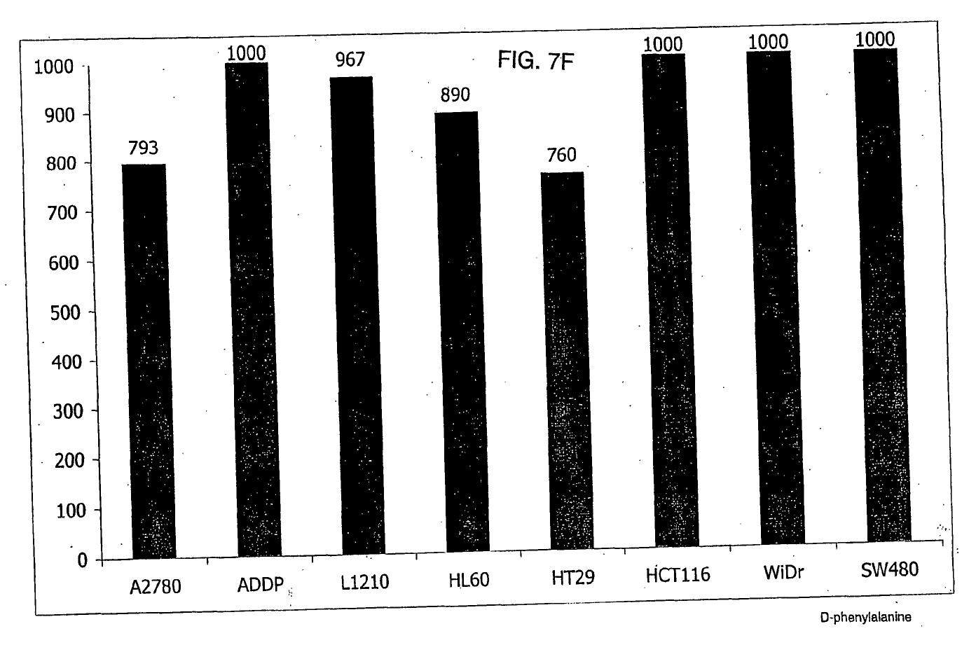

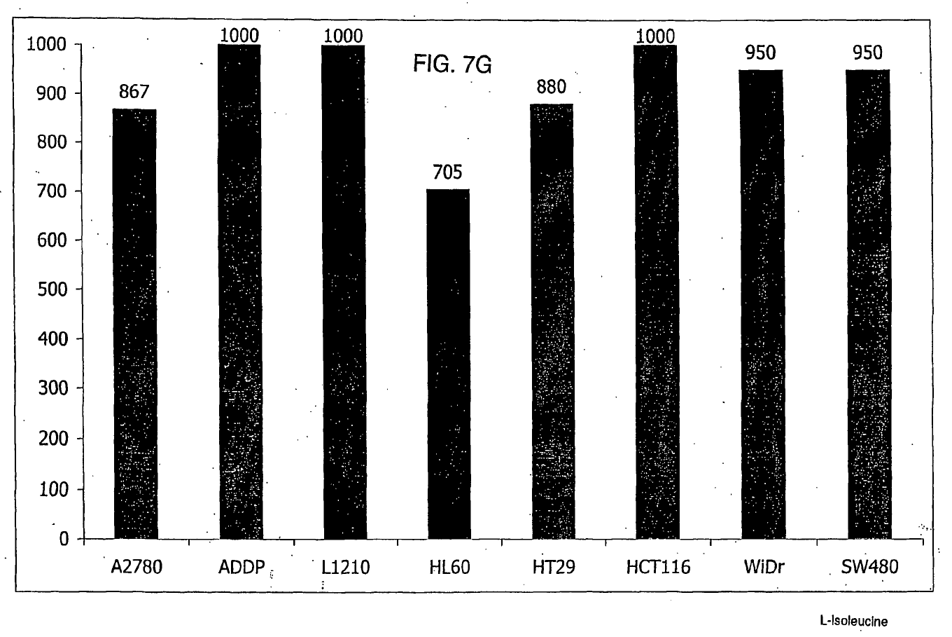

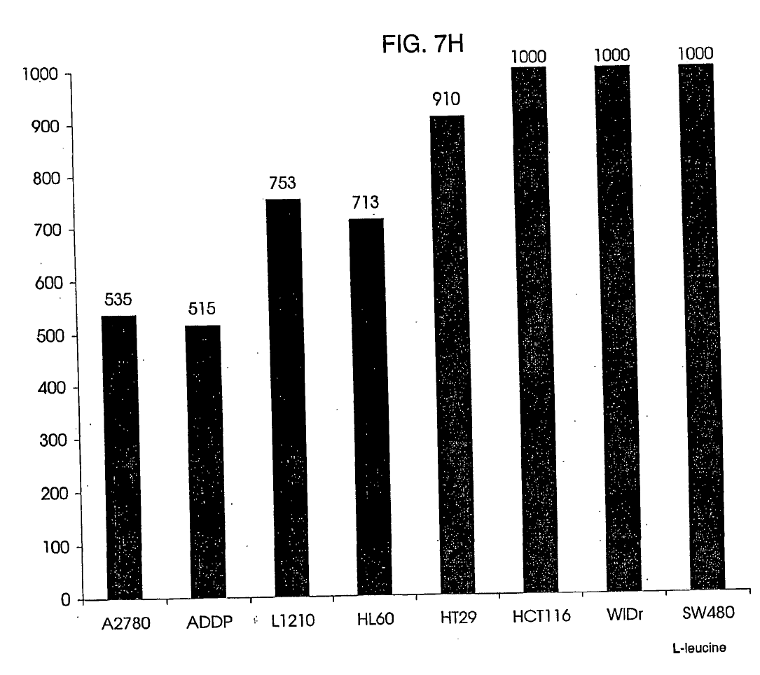

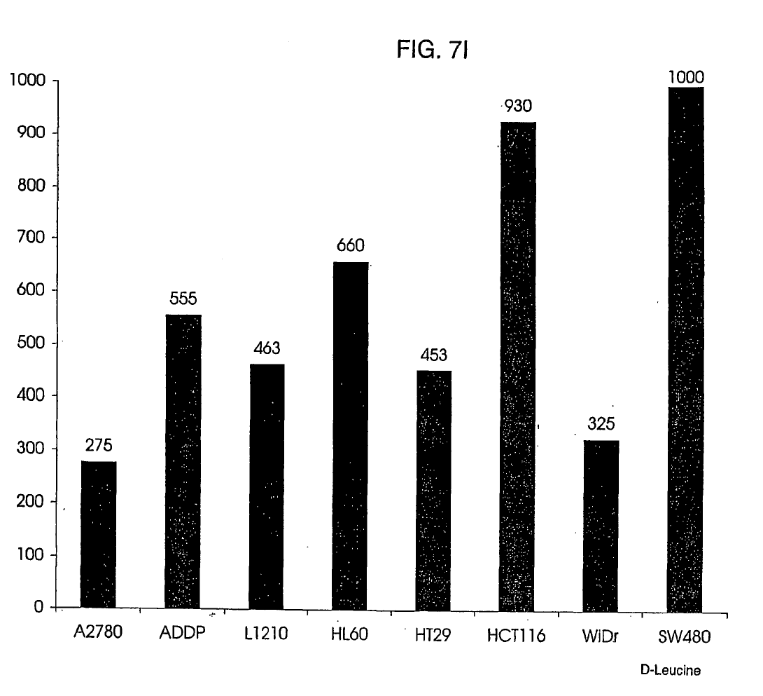

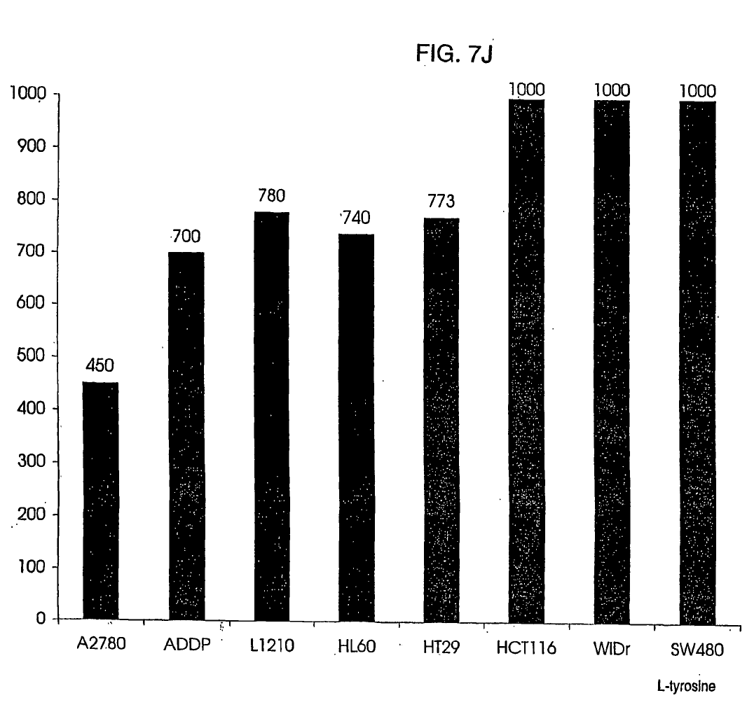

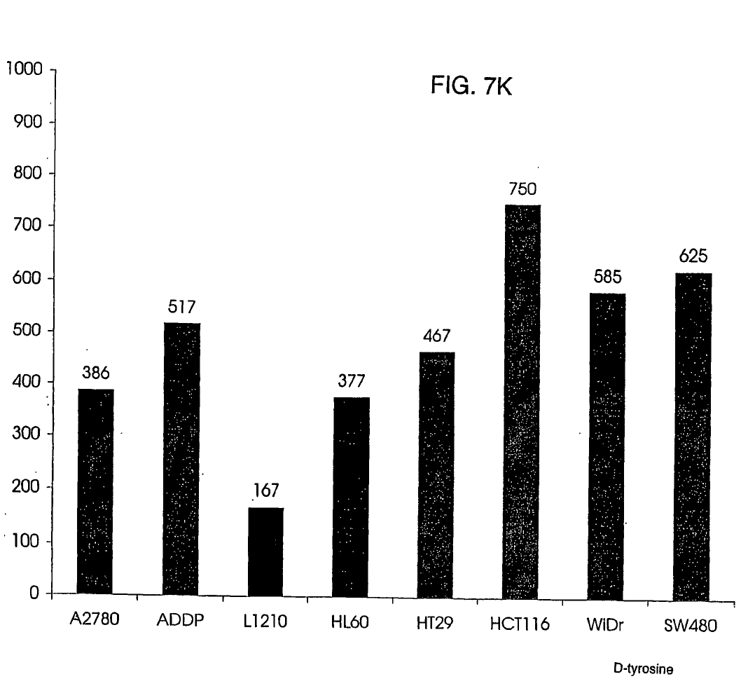

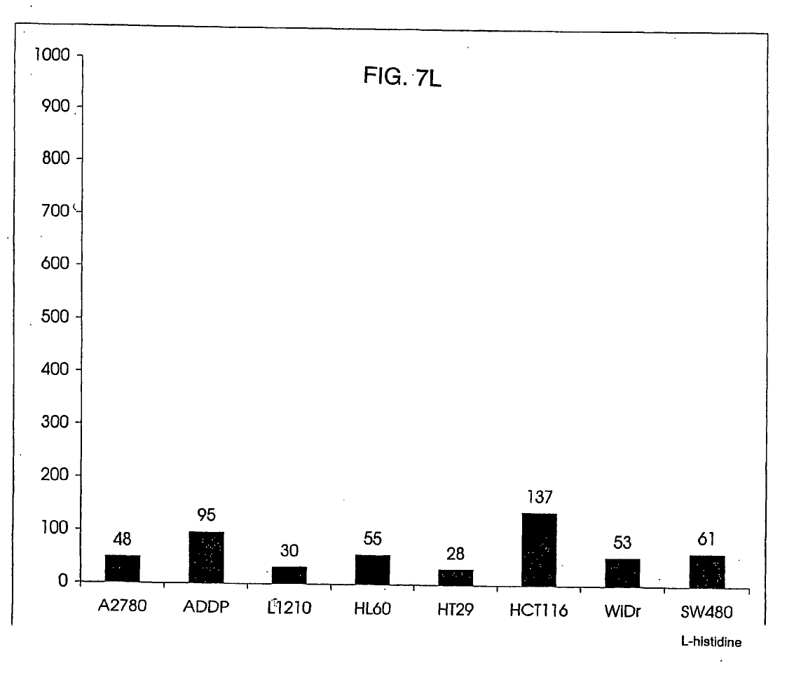

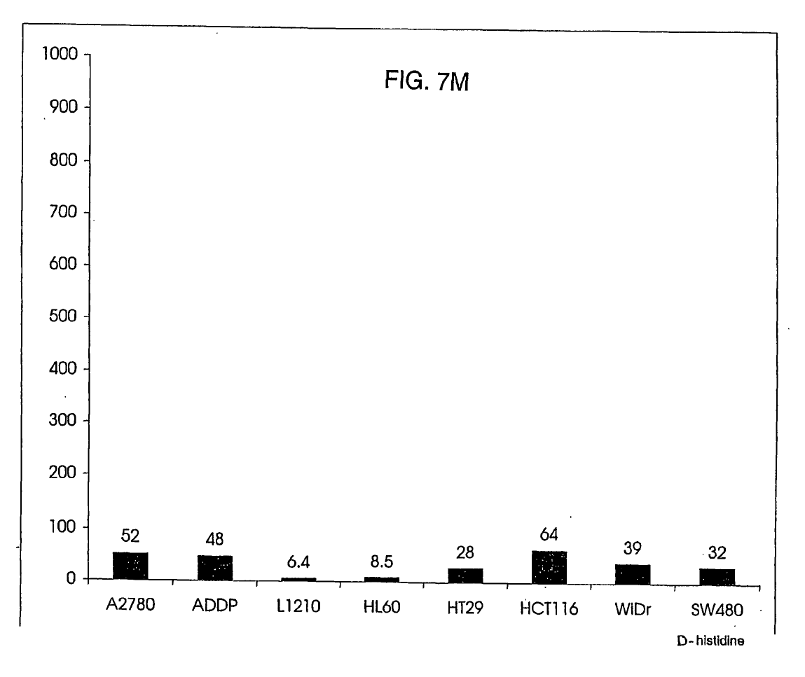

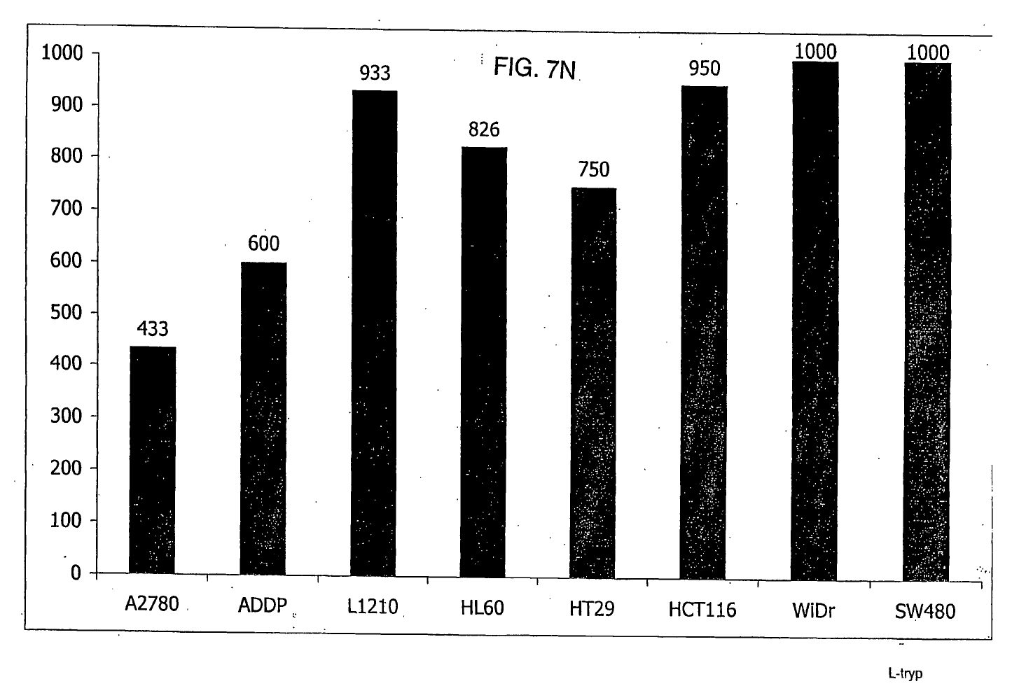

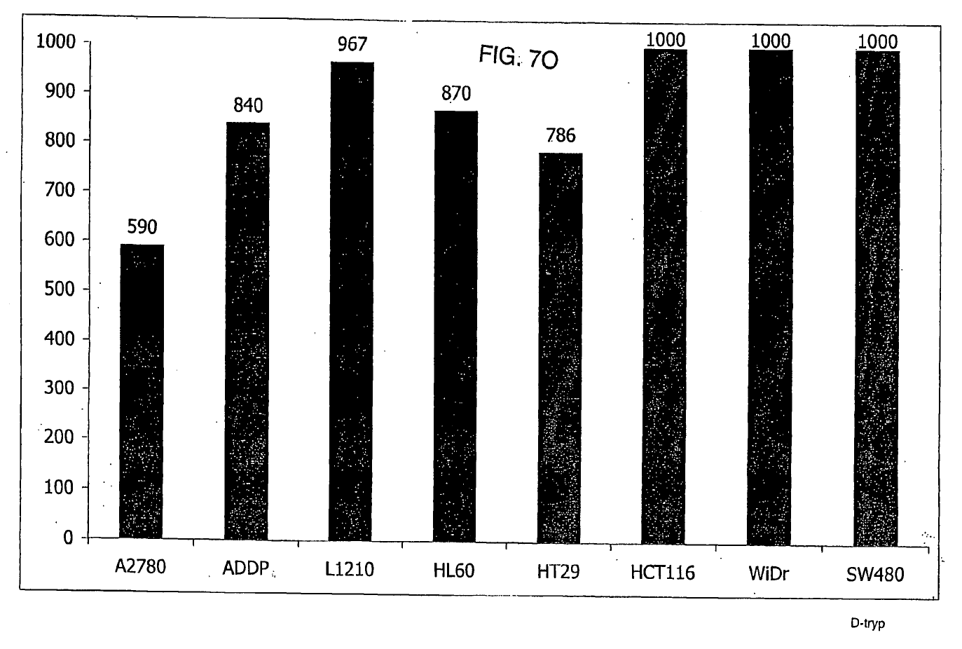

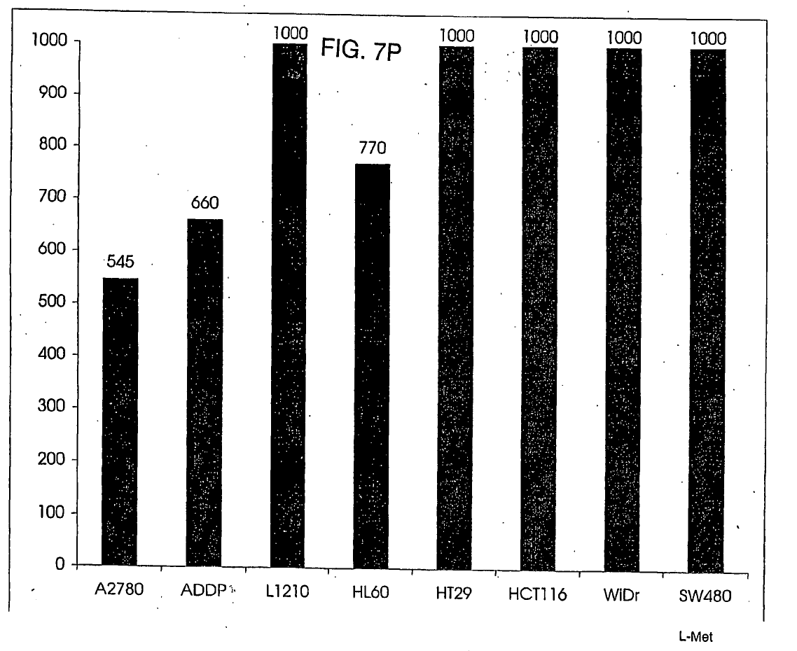

Results of cytotoxicity studies using several different cell types are shown in FIG. 7.

### EXAMPLE 6

Cell Cycle Events

By convention, cancer cells have an inherent abnormality in their ability to replicate and divide a process described as the cell cycle. Cell cycle progression is tightly regulated to ensure the integrity of the genome. During cell division it is imperative that each stage of the cell cycle be completed before entry into the next, and this is achieved through a series of checkpoints. Most conventional anticancer drugs and radiation therapies mediate their effects by influencing key components of the cell cycle, particularly DNA synthesis and mitosis. Such a strategy means that only the more rapidly dividing cells are targeted leaving non-dividing healthy somatic cells unaffected. An unfortunate side-effect to this strategy is that various healthy cell populations such as the bone marrow are also targeted resulting in potential adverse effects. Nonetheless, cell cycle machinery remains the primary target for Medical and Radiation Oncologists.

We have shown that the parental compound, cantharidin, also mediates its effects via the cell cycle. However, the mechanism is different to most other therapies as cantharidin was shown to bypass the G₁/S phase checkpoint and thus accelerate movement through the S-phase (DNA synthesis) of the cell cycle (FIG. 6). Although this would seem counter productive in the treatment of cancer cells, the accelerated movement into the synthesis phase was only transient (within 6 h, 25 μM) and subsequently followed by cell cycle arrest at the G₂/M interface (24 h) and cell death.

The distribution of cells into the various phases of the cell cycle can be analysed by measuring the DNA content of the cells, via propidium iodide staining and subsequent flow cytometric analysis. Thus senescent cells (G₀) and cells in the first gap (G₁) of the cell cycle will have approximately half of the DNA content of cells about to under go mitosis (G₂+M), while those cells synthesising DNA (S-phase) will have an intermediary DNA content. Using this technology, the effect of these new drugs on cells in the various phases of the cycle can be determined.

While such a methodology allows for determination of the distribution of cells within each phase of the cell cycle, particularly the premature accumulation of cells into the S-phase, it does not allow differentiation between accumulation as a result of accelerated movement (checkpoint abrogation) or simply a blockage in DNA synthesis as observed when using agents that inhibit DNA synthesis.

In order to differentiate between these phenomena, the ³H-thymidine uptake assay can be employed. This assay will determine whether an accumulation of cells in the S-phase is a result of accelerated movement through the cell cycle as evidenced by an increase in DNA synthesis or rather a result of a blockage in DNA synthesis. We have shown that cantharidin also induces a G₂/M phase cell cycle blockage. However, cell cycle analysis cannot differentiate between the accumulation of cells in either the G₂ (second gap) or M (mitotic) phases of the cell cycle. In order to further clarify the effect of these new agents on mitotic events of the cell cycle the cells are examined histologically for the various stages of mitosis via fluorescent labelling of DNA (Hoechst staining), spindles, and kinetechores. This techniques allows for determination as to whether the cells are undergoing premature mitosis, whether the chromosomes are equatorially aligned for mitosis, and whether adequate spindle formation has been achieved. Abnormalities in these stages together with abnormal cell cycle movement have been described for other phosphatase inhibitors including fostriecin, okadaic acid, calyculin A, microcystin-LR, and tautomycin.

Thus, it will be clear to the skilled addressee that ³H-thymidine uptake studies can be conducted to definitively identify the ability of the protein phosphatase inhibitors to accelerate movement through the cell cycle. Previous studies of cantharidin have shown that this acceleration is transient and detectable only within 6 h after treatment.

### EXAMPLE 7

Combination Studies

Most drug discovery has focused on the development of new single agents. However, in light of the success of combination chemotherapy it is increasingly apparent that successful anticancer treatment of the future will be based upon the discovery of agents that act in concert either to prevent development of resistance, or are ‘synergistic’ in their action (SA32). In this context, we have examined the effect of combining cantharidin with the antifolate drug, Thymitaq.

Thymitaq is representative of a large range of conventional chemotherapy drugs that are the first line treatment of many cancers and that deplete the supply of thymidylate which is critical for DNA synthesis. In these studies the Median Effect method was utilised to determine whether the combination index (CI) of these two agents was either antagonistic (CI>1), additive (CI=1), or synergistic (CI<1), after a total of 72 h exposure (SA32).^{xxii}. A sequential drug regime was also examined as cantharidin was observed to induce cellular effects quicker than Thymitaq. Thus, the sequential exposure of HT29 cells to Thymitaq followed by cantharidin 24 h later produced a CI of 0.52 indicating a strong synergistic cytotoxic interaction. Other protein phosphatase inhibitors such as okadaic acid and calyculin A have been shown to enhance chemotherapy and radiation treatment respectively. Thus, it will be clear to the skilled addressee that the compounds of the present invention are likely to be suitable for combination therapy with, for example, more conventional treatments including radiation, cisplatin, 5-flouracil, methotrexate, thymitaq and taxol.

Although the invention has been described with reference to specific examples, it will be appreciated by those skilled in the art that the invention may be embodied in many other forms.

### References and Notes

i. Wang G-S. Medical uses of mylabris in ancient china and recent studies. J. Ethnopharmacol. 1989, 26, 147-162.

ii. Scheithauer et al., 1986

iii.Roberge M; Tudan C; Hung S M; Harder K W; Jirik F R. Antitumour drug fostriecin inhibits the mitotic entry checkpoint and protein phosphatases 1 and 2A. Cancer Res. 1994, 54, 6115-6121.

iv. Li Y M; Mackintosh C; Casida J E. Protein phosphatase 2A and its [³H]cantharidin/[³H]endothall thioanhydride binding site. Inhibitor specificity of cantharidin and ATP analogues. Biochem. Pharm. 1993, 46, 1435-1443.

v. (a) Enz A; Zenke G; Pombo-Villar E. 7-Oxa[2.2.1]bicycloheptane-2,3-dicarboxylic acid derivatives as phosphatase inhibitors. Bioorg, Med. Chem. Lett. 1997, 7, 2513-2518; (b) Sodeoka M; Baba Y; Kobayashi S; Hirukawa N. Structure-activity relationship of cantharidin derivatives to protein phosphatases 1, 2A₁, and 2B. Bioorg. Med. Chem. Lett. 1997, 7, 1833-1836; (c) Tatlock J H; Linton M A; Hou X J; Kissinger C R; Pelletier L A; Showalter R E; Tempczyk A; Villafranca J E. Structure-based design of novel calcineurin (PP2B) inhibitors. Bioorg. Med. Chem. Lett. 1997, 7, 1007-1012.

vi. Laidley C W; Dauben W G; Guo Z R; Larn J Y L, Casida J E. 2-Carboxymethylendoathal analogues as affinity probes for stabilised protein phosphatase 2A. Bioorg. Med. Chem. 1999, 7, 2937-2944.

vii.Stein, G S. et al. The molecular basis of cell cycle and growth control. 1998, Wiley-Liss Publishers, New York.

viii.Huang X; Honkanen R E. Molecular cloning, expression, and characterisation of a novel human serine/threonine protein phosphatase, PP7, that is homologous to Drosophila retinal degeneration C gene product (rdgC). J Biol. Chem. 1998, 273, 1462-1468.

ix. Lazzereschi D; Coppa A; Mincione G; Lavitrano M; Fragomele F; Colletta G. The phosphatase inhibitor okadaic acid stimulates the TSH-induces G1-S phase transition in thyroid cells. Exp Cell Res. 1997, 234, 425-433.

x. Kawamura K-I, Grabowski D, Weizer K, Bukowski R, Ganapathi R, Br. J Cancer 1996, 73, 183-188.

xi. Nakamura K. Antoku S. Cancer Res 1994, 54, 2088-20990.

xii. Durfee et al., 1993

xiii.Carlson S G; Eng E; Kim, E C; Perlman E J; Copeland T D; Ballermann B J. Expression of SET, an inhibitor of protein phosphatase 2A, in renal development and Wilms-tumor. J. Am. Soc. Nephrology, 1998, 9, 1873-1880.

xiv.Yamashita K., et al. Okadaic acid, a potent inhibitor of type 1 and type 2A protein phosphatases, activates cdc2/H1 kinase and transiently induces a premature mitosis-like state in BHK21 cells. EMBO J 1990, 9: 4331-4338.

xv.Ghosh S; Schroeter D; Paweletz N. Okadaic acid overrides the S-phase check point and accelerates progression of G₂-phase to induce premature mitosis in HeLa cells. Exp Cell Res. 1996,227,165-169.

xvi.Gauss, C-M; Sheppeck, J E; Nairn, A C; Chamberlin, A R; A molecular modelling analysis of the binding interactions between the okadaic acid class of natural product inhibitors and the Ser-Thr phosphatases, PP1 and PP2A. Bioorg. Med. Chem. 1997, 5, 1751-1773.

xvii.Taylor C; Quinn R J; Suganuma M; Fujiki H. Inhibition of protein phosphatase 2A by cyclic peptides modelled on the microcystin ring Bioorg. Med. Chem. Lett. 1996, 6, 2113-2116, and references therein.

xviii.Goldberg, J; Huang, H-B; Kwon, Y-G; Greengard, P; Nairn, A C; Kuriyan, J. Three-dimensional structure of the catalytic subunit of protein serine/threonine phospahatase-1. Nature 1995, 376, 745-753.

xix.Sheppeck J E II; Gauss C-M; Chamberlin A R. Inhibition of the Ser-Thr phosphatases PP1 and PP2A by naturally occurring toxins. 1997, 5, 1739-1749.

xx.McCluskey, A, Walkom, C, Moran, S, Acland, S P, Gardiner, E, Sakoff, J A. Synthesis, Protein Phosphatase, Molecular Modelling analysis and Anticancer activity of the Cantharirides, Bioorg. Med. Chem. 2001, manuscript in preparation.

xxi.Volter, K E; Embrey, K J; Pierins, G K; Quinn, R J. A study of the binding interactions of calyculin A and dephosphonocalyculin A with PP1, development of a molecular recognition model for the binding interactions of the okadaic acid class of compounds with PP1. European Journal of Pharmaceutical Sciences 2001, 12, 181-194.

xxii.Corey E J; Raju N; A new general synthetic route to bridged carboxylic ortho esters. Tetrahedron Letters. 1983, 24, 5571-5574.

xxiii.Smith A B III; Yager K M; Phillips B W; Taylor T M. Asymmetric synthesis of diethyl (R)-(−)-(1-amino-3-methylbutyl)phosphonate. Org. Synth. 1997, 75, 19-29.

xxiv.Chou T C; Talalay P. Quantitative analysis of dose-effect relationship: the combined effects of multiple drugs on enzyme inhibitors. Advances in Enzyme Regulation. 1984, 22, 27-55.

1. A protein phosphatase inhibitor wherein the inhibitor is a cantharimide or an analogue thereof.

2. A protein phosphatase inhibitor according to claim 1 wherein the phosphatase is a serine/threonine specific phosphatase.

3. A protein phosphatase inhibitor according to claim 2 wherein the phosphatase is PPI or PP2A.

4. A protein phosphatase inhibitor according to claim 1 wherein the protein phosphatase inhibitor is cell perrneable.

5. A compound of formula I 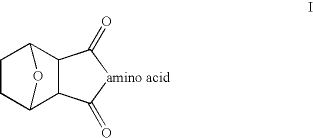
or an analogue, derivative or variant thereof.

6. A compound according to claim 5 wherein the amino acid is a D- or L-amino acid or an analogue thereof.

7. A compound according to claim 6 wherein the amino acid is selected from the group consisting of D-Alanine, L-Alanine, D-Phenylalanine, L-Phenylalanine, D-Leucine, L-Leucine, D-Isoleucine, L-Isoleucine, D-Tryptophan, L-Tryptophan, D-Histidine, L-Histidine, D-Tyrosine, L-Tyrosine, D-Glutamine and L-Methionine. More preferably, the amino acid is D-Tryptophan, L-Tryptophan, D-Histidine, L-Histidine, D-Tyrosine, L-Tyrosine, D-Glutamine or L-Methionine.

8. A compound according to claim 5 wherein the amino acid is D-Histidine or L-Histidine.

9. A compound according claim 5 wherein the compound is a cell cycle modulator.

10. A compound according to claim 9 wherein the compound stimulates the cell cycle.

11. A compound according to claim 5 wherein the compound is a protein phosphatase inhibitor.

12. A compound according to claim 11 wherein the compound is an inhibitor of PP1 and/or PP2A.

13. A method of preparing a material selected from the group consisting of a protein phosphatase inhibitor consisting of formula I, and a protein phosphatase inhibitor wherein the inhibitor is a cantharimide or an analogue thereof, comprising combining a compound of formula II 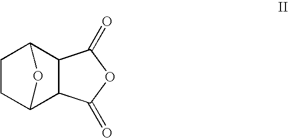
or an analogue, derivative or variant thereof, with an amino acid or an analogue, derivative or variant of an amino acid.

14. A method according to claim 13 wherein the method includes combining the compound of formula II with the amino acid in the presence of an amine.

15. A method according to claim 14 wherein the amine is a tertiary amine.

16. A method according to claim 13 wherein the amine is Et₃N.

17. A method according to claim 13 wherein the method further includes combining the compound of formula II with the amino acid in the presence of an organic solvent.

18. A method according to claim 17 wherein the organic solvent is PhCH₃.

19. A method according to claim 13 wherein the compound of formula II or an analogue, derivative or variant thereof is combined with the amino acid or an analogue, derivative or variant of an amino acid at a temperature between 15 and 300° C.

20. A method according to claim 19 wherein the temperature is room temperature.

21. A method according to claim 20 wherein the temperature is between 100° C. and 250° C.

22. A method according to claim 21 wherein the temperature is approximately 200° C.

23. A method according to claim 13 wherein the protein phosphatase inhibitor or the compound of formula I is further purified.

24. A method according to claim 23 wherein the protein phosphatase inhibitor or the compound of formula I is washed.

25. A method according to claim 24 wherein the protein phosphatase inhibitor or the compound of formula I is washed with NaHCO₃.

26. A method according to claim 23 wherein the protein phosphatase inhibitor or the compound of formula I is extracted.

27. A method according to claim 26 wherein the protein phosphatase inhibitor or the compound of formula I is extracted with CH₂Cl₂.

28. A method according to claim 23 wherein the protein phosphatase inhibitor or the compound of formula I is acidified.

29. A method according to claim 28 wherein the protein phosphatase inhibitor or the compound of formula I is acidified with concentrated HCl.

30. A method according to claim 23 wherein the protein phosphatase inhibitor or the compound of formula I is extracted with an organic solvent.

31. A method according to claim 30 wherein the organic solvent is ethyl acetate.

32. A method according to claim 23 wherein the protein phosphatase inhibitor or the compound of formula I is further purified by chromatography.

33. A method according to claim 32 wherein the chromatography is column chromatography.

34. A compound of formula III 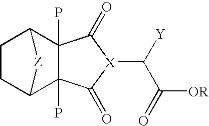
wherein Z is O, NH or NR;
X is CH or N;
wherein P may be absent or may be methyl;
Y is (CH₂)ₙ W;
wherein W is any ionisable residue and n can be any integer from 1 to 8.

35. A compound according to claim 34 wherein the compound has the compound of formula IV or the compound of formula V as follows:

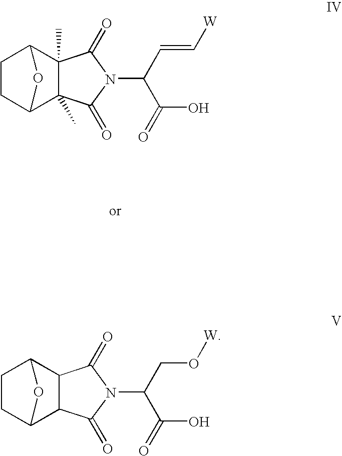

36. A compound according to claim 34 when used to modulate the cell cycle of a cell.

37. A compound according to claim 34 wherein Z is O, X is N and Y is —CH₂OW wherein W is an amino acid or an analogue or derivative of an amino acid.

38. A compound according to claim 37 wherein the amino acid is D-Histidine or L-Histidine.

39. A compound according to claim 34 wherein Z is O, X is N and Y is —CH₂═CHW wherein W is an amino acid or an analogue or derivative of an amino acid.

40. A compound according to claim 39 wherein the amino acid is D-Histidine or L-Histidine.

41. A pharmaceutical composition including a material selected from the group consisting of a protein phosphatase inhibitor wherein the inhibitor is a cantharimide or an analogue thereof, the compound of formula I, and the compound of formula III.

42. A pharmaceutical composition according to claim 41 suitable for intravenous delivery.

43. Use of a material selected from the group consisting of a protein phosphase inhibitor wherein the inhibitor is a cantharimide or an analogue thereof, the compound of formula I, and the compound of formula III in the manufacture of a medicament.

44. Use according to claim 43 wherein the medicament is for the treatment of cancer.

45. Use according to claim 44 wherein the cancer is leukemia, ovarian, colon or kidney cancer.

46. A method of treating a disorder or a disease including administering a material selected from the group consisting of a protein phosphase inhibitor wherein the inhibitor is a cantharimide or an analogue thereof, the compound of formula I, and the compound of formula III.

47. A method according to claim 46 wherein the disease is cancer.

48. A method according to claim 47 wherein the cancer is leukemia, ovarian, colon or kidney cancer.

49. A method according to claim 46 wherein the method is combined with a further treatment.

50. A method according to claim 49 wherein the method of treatment renders treated cells more sensitive to the further treatment.

51. A method according to claim 49 wherein the further treatment is a treatment for cancer.

52. A method according to claim 49 wherein the further treatment is be selected from the group consisting of radiation, cisplatin, 5-flurouracil, methotrexate, thymitaq and taxol treatment.

53. A method of regulating cell cycle progression including exposing a cell to a material selected from the group consisting of a protein phosphase inhibitor wherein the inhibitor is a cantharimide or an analogue thereof, the compound of formula I, and the compound of formula III.

54. A method according to claim 53 wherein exposure of the cell to the protein phosphase inhibitor, the compound or the pharmaceutical abrogates one or more of the cell cycle checkpoints.

55. A method according to claim 53 wherein the cell is a mammalian cell.

56. A method according to claim 53 wherein the cell is a human cell.

57. A method according claim 53 wherein the cell is a neoplastic or pre-neoplastic cell.

58. A method according to claim 57 wherein the cell is a cancer cell.

59. A method according to claim 58 wherein the cell is selected from the group of: a murine leukaemia cell, a human leukaemia cell, a human ovarian cancer cell, a cisplatin resistant cell, a human colon carcinoma cell, a human oesteosarcoma cell and a human kidney tumour cell.

60. A method according to claim 59 wherein the cell is a cell selected from those cell types known as L1210 (murine Leukaemia, p53 mt), HL60 (human leukaemia, p53 nul), A2780 (human ovarian carcinoma, p53 wt), ADD (cisplatin resistant A2780 cells, p53 mt), HCT116 (human colon carcinoma, p53 wt), SW 480 (human colon carcinoma, p53 wt), WiDr (human colon carcinoma, p53mt), HT29 (human colon carcinoma, p53 mt), 143BTK-(human oesteosarcoma) and G-401 (human kidney, Wilms tumour) cells.

61. A method of sensitising a patient to a cancer treatment, comprising administering to the patient an effective amount of a material selected from the group consisting of a protein phosphatase inhibitor wherein the inhibitor is a cantharimide or an analogue thereof, the compound of formula I, and the compound of formula III.

62. A method of preparing a compound of formula III including combining 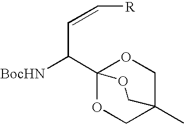
wherein R is an ionisable residue
with cantharidin or an analogue or derivative thereof.

63. A method of preparing a compound of formula III including combining 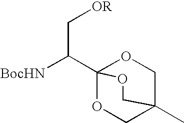
wherein R is an ionisable residue
with cantharadin or an analogue or derivative thereof.

64. A method according to claim 62 wherein the ionisable residue or amino acid analogue is a histidine analogue with a modified sidechain.

65. A method according to claim 64 wherein the modified sidechain is selected from one of the following: 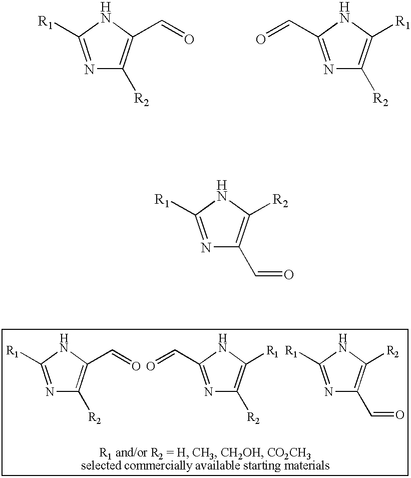
where R₁ and/or R₂═H, CH₃, CH₂OH, CO₂CH₃.

66. A method according to claim 13 wherein the method is carried out in accordance with the Horner-Emmons-Willing or Julia-olefination synthetic methodology.

67. A method according to claim 62 wherein the method is carried out in accordance with the Horner-Emmons-Willing or Julia-olefination synthetic methodology.
